(19) United States
(12) Patent Application Publication
(10) Pub. No.: US 2004/0220679 A1
(43) Pub. Date: Nov. 4, 2004

(54) Hybrid ceramic composite implants

(76) Inventors: Robert L. Diaz, Palm Beach Gardens, FL (US); Drew D. Ehlert, Palm Beach Gardens, FL (US); David Roy Campbell, Jupiter, FL (US)

Correspondence Address: Michael A. Slavin, McHale & Slavin, P.A., Suite 402, 4440 PGA Boulevard, Palm Beach Gardens, FL, 33410, US

(21) Appl. No.: 10/429,201

(22) Filed: May 1, 2003

(51) Int. Cl. A61F 2/30

(52) U.S. Cl. 623/23.4

(57) ABSTRACT

A prosthesis for total or partial replacement of an articulating skeletal joint has two opposed bearing surfaces. The opposed bearing surfaces are made of a ceramic and a metal, respectively. The ceramic material is a silicon nitride or silicon carbide and the metal is preferably a nitrogen alloyed chromium stainless steel, know as Cronidur 30.

### BACKGROUND OF THE INVENTION

1. Field of the Invention

This invention relates to the field of orthopedic surgery and, particularly, to bone and joint prosthesis.

2. Description of the Prior Art

The prior art is replete with artificial bone and joint devices for surgical implantation to structurally compensate for diseased, damaged or missing natural skeletal anatomical elements. Such prosthesis include bone pins, articulating joints, both total and partial, for the knee and hip, as well as, fingers, toes, elbows, shoulders and vertebra. In total replacement both bearing surfaces of an articulating joint are artificial, eg., the femur head and the acetabulum within which the ball rotates.

These prosthesis are made from a variety of biologically inert materials having the requisite strength and longevity to provide the recipient with approximately normal activity and life style. Another important consideration in selecting materials and design is to reduce or eliminate repeated surgeries during the recipient's life. For many years, the standard material has been stainless steel and other metal alloys, such as cobalt-chromium-molybdenum, for such prosthesis. For example, U.S. Pat. No. 5,263,988 to Huebner discloses a hip prosthesis made from the cobalt alloy.

Another steel is known as nitrogen alloyed class III super 12 chromium stainless steel, called Cronidur 30, as disclosed in German application, DE 19729450 C2, published Dec. 9, 1999, and produced in Germany by Vereinigte Schmiedewerke GmbH. This material is used as a hard bearing surface in space shuttle engine turbopumps. As a component of space shuttle main engines, this steel has undergone intense testing and review. Some of the operational requirements include reliability in sustaining high hertzian contact stresses at high speeds in liquid oxygen or hydrogen under marginal lubrication conditions. Between missions the steel must resist stress corrosion cracking at ambient conditions with periods of high humidity. These requirements necessitate a steel with high hardness (58 Rc), fracture toughness, corrosion resistance, and liquid oxygen comparability. The prior art benchmark has been AISI 440C martensitic stainless steel however this steel has a limited fracture toughness and modest stress corrosion resistance. Cronidur 30 was developed to overcome these weaknesses and is a martensitic stainless steel based on nitrogen alloying to a chromium stainless steel. The steel exhibits superior corrosion resistance with a surface hardness of 58-60 Rc and core fracture toughness in excess of 50 ksi/in. The microstructure consists of a fine dispersion of refractory metal carbides in a martensitic matrix that affords the alloy a good balance of strength and toughness. In contrast, AISI 440C comprises of coarse primary carbide stringers in a martensitic matrix. These same properties that make the Cronidur 30 desirable in the space program also make the steel preferable in the surgical implant field.

More recently, non-metals such as polymers and ceramics, have been gaining acceptance in the field. Some flexible hinge joints use silastic material or a hard bearing surface laminated to a ultrahigh molecular weight polyethylene. U.S. Pat. No. 6,524,342 to Muhlhauser discloses a shoulder joint that has components of metal and others of ceramic materials. Pope et al, U.S. Pat. No. 6,517,583, disclose hip joints and knee joints with hardened bearing surfaces which include ceramic material or diamond material.

There are certain areas of concern when using the prior art components for prosthesis. Osteolysis from particulate debris in the artificial joint can cause complications for the recipient of the prosthesis. Inflammation may result from the generation of large amounts of small wear particles and in acute cases the implant may fail due to lack of stability in the bone/prosthesis connection or undue wear in the bearing surface. The metal to metal prosthesis also generate particles within the joint, though to a lesser degree. In spinal arthroplasty, wear particles may cause a severe inflammatory response, with resultant nerve root adhesions.

Silicon nitride forms the basis for ceramic products of general utility, as taught by Ikeda et al, U.S. Pat. No. 5,635,431, Goto et al, U.S. Pat. No. 4,886,767, and Fukuhara et al, U.S. Pat. No. 4,609,633. This sintered material is extremely hard and highly resistant to abrasion. As disclosed by Goto et al, the material may be cast or molded. A preferred silicon nitride is disclosed in U.S. Pat. No. 4,986,972, U.S. Pat. No. 4,911,870, and U.S. Pat. No. 4,902,653 which has the desired characteristics of hardness, toughness, and resistance to abrasion.

What is needed in the art is a prosthesis with reduced friction and wear on the bearing surfaces and reduced incidence of osteolysis.

### SUMMARY OF THE INVENTION

A prosthesis for total or partial replacement of an articulating skeletal joint having two opposed bearing surfaces. The opposed bearing surfaces are made of a ceramic and a metal, respectively. The ceramic material is a silicon nitride or carbide and the metal may be a nitrogen alloyed chromium stainless steel, know as Cronidur 30.

Thus, an objective of this invention is to teach the use of materials for the bearing surfaces in skeletal prosthesis with improved surface smoothness and wear resistence to reduce the causes of osteolysis.

Another objective of this invention is to teach the use of a silicon nitride or carbide sintered body as a bearing surface in a prosthesis.

A further objective of this invention is to teach the use of a ceramic bearing surface in contact with a metal bearing surface in a prosthesis.

Yet another objective of this invention is to teach the combination of a silicon nitride or silicon carbide bearing surface and a nitrogen alloyed chromium steel bearing surface in contact with each other in an articulating prosthesis.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

FIG. 1 is a side view, partially in section, of a prior art total hip prosthesis;

FIG. 2 is a side view, partially in section, of another prior art total hip prosthesis;

FIG. 2A is a side view, partially in section, of another prior art total hip prosthesis;

FIG. 3 is a side view of a artificial head of the femur of this invention;

FIG. 4 is a top view of the head of the femur of FIG. 3;

FIG. 5 is a cross section of the artificial acetabulum of this invention; and

FIG. 6 is a top view of the acetabulum of FIG. 5.

### DETAILED DESCRIPTION OF THE INVENTION

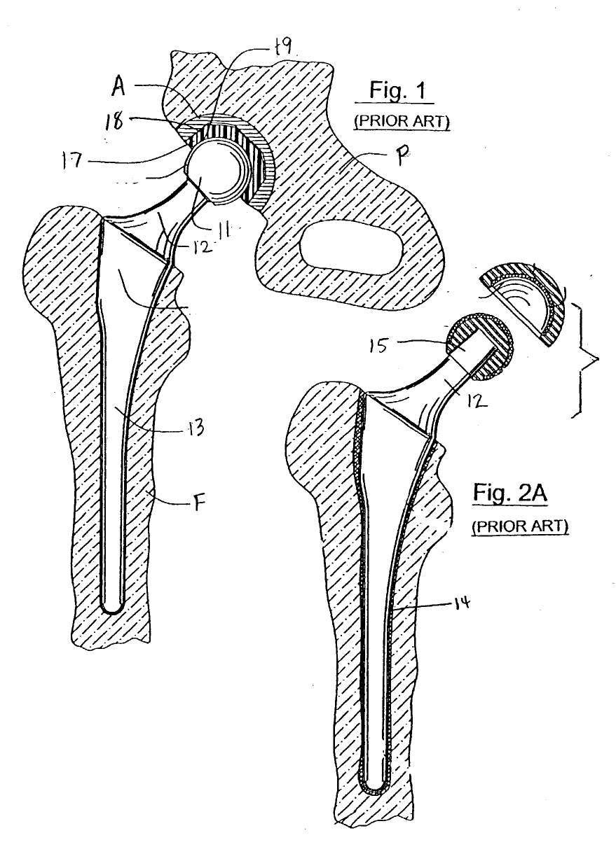

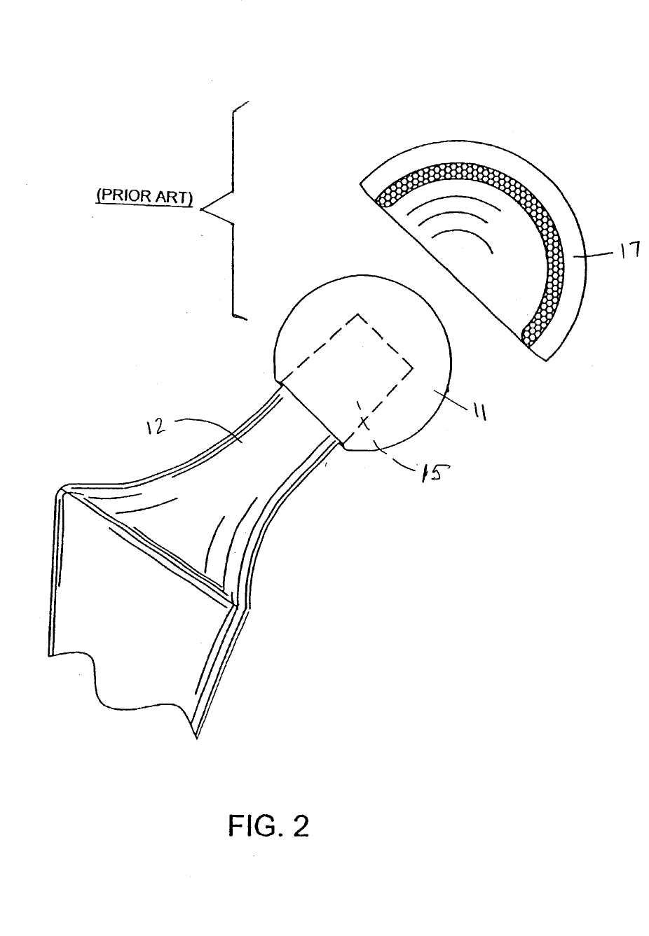

FIGS. 1, 2 and 2A depict prior art hip prosthesis showing the pelvis P, a proximal acetabulum A and distal femur F components forming a total hip replacement. The head of the femur F has been replaced with a ball 11 mounted on a neck 12 which is supported by a rod 13. The rod 13 is inserted in a prepared bore in the central canal of the femur. The orientation of the ball, neck and rod is adjusted to simulate the angular disposition of the natural head of the femur to assure proper rotation in the acetabulum A. The rod 13 may have a outer surface modified to make a better connection with the shaft of the femur.

In FIG. 2A, a coating 14 is shown to improve the surface area between the natural bone and the prosthesis. Also, the bore and rod may have an intermediate layer of bone cement, growth factors or combinations thereof (not shown).

The ball 11 may be a unitary construction with the neck, as shown in FIG. 1, or it may be integrally mounted on a pin 15, as shown in FIG. 2. Also shown in FIG. 2A is an integrally mounted ball 11 with a coating or layer of a different material 16 on the outer surface.

The prosthetic acetabulum 17, as shown in FIG. 1, is a cup shaped structure that is attached to the pelvis P by screws (not shown). The cup shaped structure 17 is illustrated with a smooth domed outer surface engaging a prepared surface in the natural bone. The interior surface 18 of the dome may be shaped to receive a liner 19 having an outer surface keyed to the inner surface of the cup shaped structure 17. The interior surface of the liner 19 must complement the shape of the ball 11 for satisfactory performance. As shown in FIGS. 1 and 2, the exterior of the ball 11 and the interior of the liner may be of different materials or, as shown in FIG. 2A, the complementary surfaces may be the same.

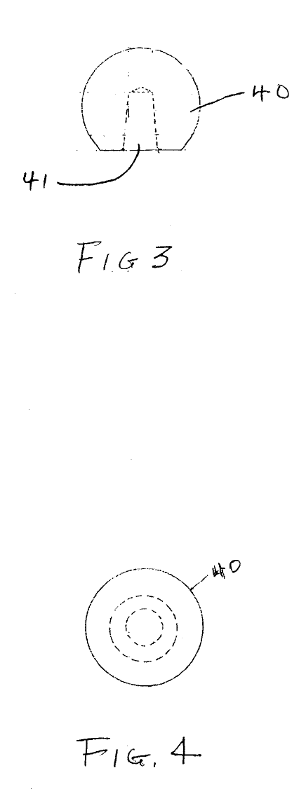

The prosthetic ball 40, shown in FIG. 3, is preferably made from a silicon nitride or silicon carbide material. The ball 40 may be formed entirely of the ceramic material with a bore 41 for mounting on the pin of a femoral rod. The silicon nitride or silicon carbide material can be hot isostatic pressed formed substantially in the final shape. The ball 40 is then polished to a mirror-like finish that will fit over the prosthetic pin, such as pin 15. The ball and the pin are permanently affixed with each other. The ceramic ball is completely corrosion resistant and is non-abrasive. The solid matrix eliminates the wear particles, such as liberated from metal, coated metal and polyethylene implants. The ball 40 has excellent thermal conductivity thereby reducing patient discomfort associated with exposure to cold weather. Further, the silicon nitride implant will react well with x-ray and MRI (magnetic resonance imaging) diagnostic procedures. The rod, neck and pin of the prosthesis may be of any materials that have the requisite biological non-toxicity and strength, including metals and polymers.

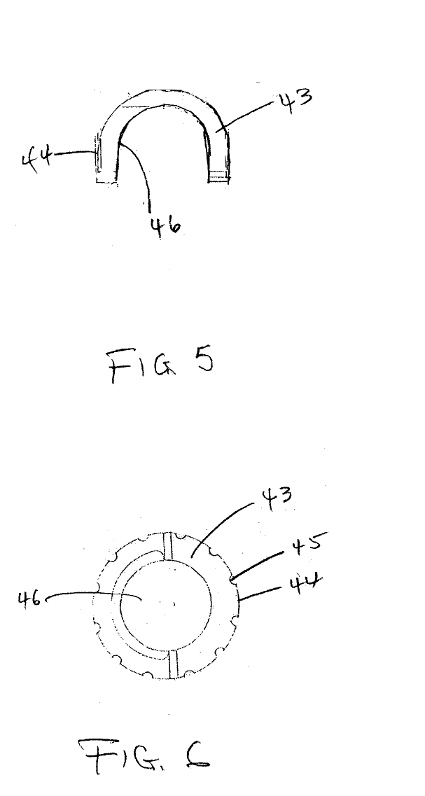

The acetabulum 43, shown in FIG. 5, is preferably constructed of Cronidur 30 steel, described above. The wear characteristics of this material are especially suited to use with the silicon nitride of the ball to reduce accumulation of wear particles in a prosthesis. This material is preferred because it possesses superior hardness, high wear resistance, good fracture toughness and excellent corrosion resistance. While other acceptable steels may have one or more of these properties, none have all of these properties plus the corrosion resistance of Cronidur 30. The acetabulum may be made entirely of Cronidur 30 or only the concave surface.

The outer or convex surface of the acetabulum 43 may have lands 44 and grooves 45 forming a fluted surface for increasing the resistance to turning in the pelvis. The acetabulum may be driven into the bone to seat the lands and grooves to the bone. Bone screws may also be used in addition to or in lieu of the fluting.

The highly polished concave surface 46 of the acetabulum 43 serves as a bearing surface for the rotation of the silicon nitride ball 40. The strength and hardness of the steel matches the properties of the silicon nitride or silicon carbide to lessen the collection of wear particles in a joint. While the invention has been described in relation to a total hip replacement, the silicon nitride or silicon carbide material may be used in total or partial replacement of other articulating joints, such as the knee, shoulder, vertebrae or others. Alternative constructions would include fabrication of the entire prosthesis from either of the materials of this invention, coating different metallic or polymeric materials with the materials of this invention, to include coating the Cronidur 30 with the silicon nitride, and reversing the use of the materials on components. The invention has been described in relation to a total hip replacement however, the invention may be used in prosthesis for all other articulation joints of the body it should be considered a teaching for use with all skeletal joints.

A number of embodiments of the present invention have been described. Nevertheless, it will be understood that various modifications may be made without departing from the spirit and scope of the invention. Accordingly, it is to be understood that the invention is not to be limited by the specific illustrated embodiment but only by the scope of the appended claims.

What is claimed is:

1. A prosthesis for implantation into bone to provide a bearing surface between contacting elements of a skeleton, said prosthesis comprising an attachment means for fixedly attaching said prosthesis to one element of said skeleton, a bearing surface supported by said attachment means for contacting another element of said skeleton, said bearing surface being made of a material having sufficient hardness, toughness and abrasion resistance to prevent accumulation of wear particles.

2. A prosthesis of claim 1 wherein said bearing surface is made of a silicon nitride or silicon carbide.

3. A prosthesis of claim 1 wherein said bearing surface is made of a nitrogen alloyed chromium stainless steel.

4. A prosthesis for implantation in an articulating joint of a skeleton comprising a distal component having a first bearing surface and a proximal component having a second bearing surface, said first and second bearing surfaces in relatively movable contact, one of said first and second bearing surfaces being formed of a ceramic material and the other of said first and second bearing surfaces being a biologically inert material whereby relative movable contact between said first and second bearing surfaces results in little or no accumulation of wear particles.

5. A prosthesis of claim 4 wherein said ceramic material is silicon nitride or silicon carbide.

6. A prosthesis of claim 4 wherein said other of said first and second bearing surfaces is a nitrogen alloyed chromium stainless steel.

7. An prosthesis of claim 5 wherein said other of said first and second bearing surfaces is a nitrogen alloyed chromium stainless steel.

8. A prosthesis of claim 4 wherein said distal component includes a rod for insertion into a bone on one side of said articulating joint, said rod terminating in said first bearing surface, said proximal component includes attachment structure for fixing said proximal component to a bone on the other side of said articulating joint, said attachment structure terminating in a second bearing surface, said first and second bearing surfaces in relatively movable contact, said relatively movable contact producing no wear particles in said articulating joint.

9. A prosthesis of claim 8 wherein said distal component has a rod, an intermediate neck and terminates in a ball, said ball forming said first bearing surface and made of a silicon nitride or silicon carbide.

10. A prosthesis of claim 9 wherein said neck includes a pin and said ball has a bore, said pin seated within said bore and fixed thereto.

11. A prosthesis of claim 8 wherein said proximal component is in the form of a dome with a highly polished concavity forming said second bearing surface, said convex surface forming said attachment structure, said second bearing surface being a nitrogen alloyed chromium stainless steel.

12. A prosthesis of claim 9 wherein said proximal component is in the form of a dome with a highly polished concavity forming said second bearing surface, said convex surface forming said attachment structure, said second bearing surface being a nitrogen alloyed chromium stainless steel, said concavity having complementary dimensions of said ball.
